(19) United States
(12) Patent Application Publication
(10) Pub. No.: US 2026/0284430 A1
(43) Pub. Date: Sep. 24, 2026

(54) MOTION-TRACKING AND BLUR COMPENSATION FOR LASER TREATMENT DEVICE

(71) Applicant: Avava, Inc., Waltham, MA (US)

(72) Inventors: Jayant Bhawalkar, Auburndale, MA (US); Rajender Katkam, Boston, MA (US); Vincent Zuo, Reading, MA (US)

(21) Appl. No.: 19/474,691

(22) PCT Filed: Apr. 8, 2024
(86) PCT No.: PCT/US2024/023568

§ 371 Date: Oct. 10, 2025

Related U.S. Application Data
(60) Provisional application No. 63/458,520, filed on Apr. 11, 2023.

(51) Int. Cl. A61N 5/067; A61N 5/06

(52) U.S. Cl. CPC A61N 5/067; A61N 5/0616; A61N 2005/0626; A61N 2005/066

(57) ABSTRACT

Treatment devices and related methods are provided. The treatment devices include a handpiece configured to deliver electromagnetic radiation (EMR) along an optical axis, a window disposed at an end of the handpiece and intersecting the optical axis, an optical arrangement disposed within the handpiece, an EMR source in communication with the handpiece and configured to emit a treatment radiation through the optical arrangement and the window along the optical axis, and at least one light source disposed in the housing and offset from the optical axis. The window is configured to directly contact tissue. The optical arrangement includes a high NA lens. The at least one light source is configured to illuminate the contacted tissue at a substantially oblique angle and is configured to emit light at a wavelength at a variable wavelength range between about 300 nm and about 650 nm based on a tissue marker in the tissue.

### BACKGROUND

Lasers and light-based therapeutic devices are widely used in various medical fields, including in the field of dermatology, to deliver optical energy to target tissue. This delivery of optical energy can alter tissue, or chromophores present in tissue, through several mechanisms, including photothermal and photochemical mechanisms, and these alterations have been harnessed for well-known laser/light-based procedures, including laser hair removal, tattoo removal, removal of pigmented lesions and vascular lesions, tissue tightening, reduction in wrinkles, etc. Such devices in dermatology, for example, typically include a handpiece which is manually moved across a patient's skin by a physician. These devices deposit optical energy in short bursts to create fractional patterns comprising numerous microthermal zones (MTZ) of tissue damage that leave the majority of the skin tissue intact and untreated in order to improve a healing response of the treated tissue regions. The delivery of too little optical energy to the treatment area can result in an ineffective treatment, while the delivery of too much optical energy can result in undesirable tissue damage, e.g., burns. Further, the delivery of energy while the handpiece is moved across the patient's skin can result in the creation of fractional patterns that are less than ideal, such as fractional patents that appear smeared or blurred when compared to an ideal fractional pattern, resulting in both the reduction of energy density and increase in size of each MTZ, ultimately leading to a decreased treatment efficacy. It is therefore desirable that a treatment system and method be made available that addresses these limitations and challenges.

### SUMMARY

Embodiments for laser-based treatment devices and methods of treatment are described. In an embodiment, a treatment system is provided. The treatment system can include a handpiece having a window disposed at an end thereof, a first electromagnetic radiation (EMR) source in communication with the handpiece, a second EMR source disposed within the handpiece, an optical arrangement disposed within the handpiece, a first galvanometer in communication with the optical arrangement, and a controller in communication with the handpiece and the first EMR source. The window can be configured to directly contact tissue. The first EMR source can be configured to emit a pulsed treatment radiation along a nominal optical axis through the window. The second EMR source can be configured to illuminate contacted tissue at an illumination angle substantially oblique to the nominal optical axis. The nominal optical axis can extend through the optical arrangement. The first galvanometer can be configured to adjust a configuration of the optical arrangement. The controller can be configured to manipulate the configuration of the optical arrangement via the first galvanometer to alter an emission angle of the pulsed treatment radiation relative to the nominal optical axis and during a treatment pulse duration of the pulsed treatment radiation. The controller can also be configured to alter a wavelength of the second EMR source based on a detected tissue marker of contacted tissue.

The treatment device can vary in a number of ways. For example, the second EMR source can include a plurality of second EMR sources each at a substantially oblique angle relative to the nominal optical axis. In some variations, the plurality of second EMR sources can be disposed in a substantially ring-shaped geometry surrounding the nominal optical axis. In other variations, each of the plurality of second EMR sources can be configured to emit light at a discrete wavelength, and at least two sources in the plurality of second EMR sources can be configured to emit light at different wavelengths. In another example, the optical arrangement can include an imaging lens configured to receive backscattered light emitted by the second EMR source reflected off the contacted tissue. In some variations, the controller can be configured to identify the tissue marker based on the received backscattered light. In other variations, the imaging lens can include an adjustable iris configured to optimize an f-number of the imaging lens. In still other variations, the f-number of the imaging lens can range between about 5.2 and 8.2. In further variations, at marginal focus and quarter wave of spherical aberration, 4λ(F/#)² can be approximately equal to between 50 μm-120 μm, wherein: 2 is the one or more discrete wavelengths in nm, and (F/#) is the f-number of the imaging lens. In another example, the optical arrangement can include a high NA lens having an NA of 0.3 or greater. In yet another example, the first galvanometer can be configured to manipulate the optical arrangement to adjust the emission angle based on a velocity of the window relative to the contacted tissue. In some variations, the first galvanometer can be configured to manipulate the optical arrangement to adjust the emission angle in at least two degrees of freedom. In a further example, the treatment system can include a second galvanometer in communication with the optical arrangement. The first galvanometer can be configured to adjust the configuration of the optical arrangement to alter the emission angle about a first axis, and the second galvanometer can be configured to adjust the configuration of the optical arrangement to alter the emission angle about a second axis. The first axis can be substantially perpendicular to the second axis.

In another embodiment, a treatment system is provided. The treatment system can include an elongate housing defining a central longitudinal axis, an EMR source operably coupled to in the elongate housing and configured to emit a treatment beam toward the window, at least one light source disposed in the elongate housing and radially offset from the central longitudinal axis, an optical arrangement disposed within the elongate housing, a first galvanometer in communication with the optical arrangement, and a controller in communication with EMR source and the first galvanometer. The elongate housing can include a window disposed on the housing and intersecting the central longitudinal axis, the window being configured to contact tissue during a treatment process. The treatment beam can be emitted along a treatment path that is substantially parallel to the central longitudinal axis. The at least one light source can be configured to emit a detection beam toward the window. The optical arrangement can be configured to direct and shape the emitted treatment beam. The first galvanometer can be configured to adjust a configuration of the optical arrangement. The controller can be configured to manipulate the configuration of the optical arrangement via the first galvanometer to alter an emission angle of the treatment beam relative to the longitudinal axis. The controller can also be configured to alter a wavelength of the detection beam based on a detected tissue marker of contacted tissue.

The treatment system can vary in a number of ways. For example, the wavelength can be adjustable between about 390 nm to 650 nm. In another example, the wavelength can be proportional to the detected tissue marker. In still another example, the at least one light source can include a plurality of light sources each disposed at a substantially oblique angle relative to the longitudinal axis. In some variations, the plurality of light sources can be disposed in a substantially ring-shaped geometry. In other variations, each of the plurality of light sources can be configured to emit light at a discrete wavelength, and at least two light sources in the plurality of light sources can be configured to emit light at different discrete wavelengths. In a further example, the first galvanometer can be configured to manipulate the optical arrangement to adjust the emission angle based on a velocity of the window relative to the contacted tissue. In some variations, the first galvanometer can be configured to manipulate the optical arrangement to adjust the emission angle in at least two degrees of motion. In yet a further example, the EMR source can be configured to deliver the treatment radiation in a pulse duration, and the pulse duration can range between about 3 and 10 ms. In still a further example, the treatment device can include a second galvanometer operatively coupled to the optical arrangement. The first galvanometer can be configured to manipulate the optical arrangement to alter the emission angle about a first axis, and the second galvanometer can be configured to manipulate the optical arrangement to alter the emission angle about a second axis. The first axis can be substantially perpendicular to the second axis.

In another embodiment, a method is provided. The method can include detecting first backscattered light reflected off a first portion of tissue in contact with a laser treatment device, moving the laser treatment device from the first portion of the tissue to a second portion of the tissue, detecting second backscattered light reflected off a second portion of the tissue as the laser treatment device moves from the first portion of the tissue to the second portion of the tissue, determining a velocity of the laser treatment device relative to the tissue based upon the respective initial positions and the respective secondary positions of the one or more points of interest, delivering at least one electromagnetic radiation (EMR) pulse to the tissue with the laser treatment device for a pulse duration while the laser treatment device is moving along the tissue, and adjusting, based on the determined velocity, an emission angle of the at least one EMR pulse during the pulse duration. The backscattered light can characterize respective initial positions of one or more points of interest in the first portion relative to the laser treatment device. The second backscattered light can characterize respective secondary positions of the one or more points of interest in the second portion relative to the laser treatment device. The emission angle can be defined relative to a nominal optical axis of the laser treatment device.

The method can vary in a number of ways. For example, the at least one laser pulse can deliver between about 30 and 150 mJ to the tissue during the pulse duration. In another example, the adjusting can include changing an orientation of an optical arrangement disposed in the laser treatment device using a first galvanometer. In still another example, the method can include determining a dwell time based on an amount of energy delivered to the tissue during the pulse duration and a power of the laser treatment device. In some variations, the method can include determining a velocity compensation vector based on the velocity of the laser treatment device and the determined dwell time and adjusting the emission angle based on the determined velocity compensation vector. In other variations, the velocity compensation vector can be determined between about every 50 to 100 ms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the disclosure will be more fully understood from the following detailed description taken in conjunction with the accompanying drawings, in which:

FIG. 1 is a chart depicting an absorption spectrum of melanin;

FIG. 2 is a schematic view of a treatment device according to an embodiment;

FIG. 3A is a cross-sectional view of the treatment device of FIG. 2;

FIG. 3B is a cross-sectional view of the treatment device of FIG. 2 with optical energy flowing therethrough;

FIG. 3C is a partial cross-sectional view of a snout of the treatment device of FIG. 2 with optical energy flowing therethrough;

FIG. 4 is a schematic view of optical energy flowing through an imaging channel of the treatment device of FIG. 2;

FIG. 5 is a schematic view of optical energy flowing through a tracking channel of the treatment device of FIG. 2;

FIG. 6 is a reference for a method for determining a position of one or more illumination sources usable with the treatment device of FIG. 2;

FIG. 7A is a cross-sectional view of the snout of the treatment device of FIG. 2 with a position of an illumination source used for motion-tracking determined using the reference of FIG. 6;

FIG. 7B is a partial cross-sectional view of the snout of FIG. 7A;

FIG. 7C is a simulation of illuminance at a surface of target tissue using the parameters depicted in FIG. 7A;

FIG. 8A is a cross-sectional view of the snout of FIG. 2 with a position of an illumination source used for imaging determined using the reference of FIG. 6;

FIG. 8B is a partial cross-sectional view of the snout of FIG. 8A;

FIG. 8C is a simulation of illuminance at a surface of target tissue using the parameters depicted in FIG. 8A, with an inset showing alternate positions of the illumination source;

FIG. 9 is a diagram depicting a method to determine a wavelength of one or more illumination sources, according to an embodiment;

FIG. 10 is a timing diagram of the method of FIG. 9;

FIG. 11 is a schematic drawing of blur compensation hardware usable with the treatment device of FIG. 2, with an inset depicting compensated and uncompensated treatment patterns; and

FIG. 12 is a diagram depicting a method of compensating for blur in a treatment procedure using the blur compensation hardware of FIG. 11.

It is noted that the drawings are not necessarily to scale. The drawings are intended to depict only typical aspects of the subject matter disclosed herein, and therefore should not be considered as limiting the scope of the disclosure. The systems, devices, and methods specifically described herein and illustrated in the accompanying drawings are non-limiting exemplary embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Embodiments of the disclosure are discussed in detail below with respect to fractionated treatment including skin rejuvenation and skin resurfacing, for example skin resurfacing for: acne, chickenpox and surgical scars, periorbital and perioral wrinkles, photoageing changes, facial dyschromias, and stretch marks. Additional treatments related to the disclosure include treatment of pigmentary conditions of the skin, such as melasma, and other pigmentary conditions, such as granuloma annulare.

The disclosed embodiments can be employed for treatment of other pigmentary and non-pigmentary conditions and other tissue and non-tissue targets without limit. Examples of pigmentary conditions can include, but are not limited to, post inflammatory hyperpigmentation (PIH), dark skin surrounding eyes, dark eyes, café au lait patches, Becker's nevi, Nevus of Ota, congenital melanocytic nevi, ephelides (freckles) and lentigo. Additional examples of pigmented tissues and structures that can be treated include, but are not limited to, hemosiderin rich structures, pigmented gallstones, tattoo-containing tissues, and lutein, zeaxanthin, rhodopsin, carotenoid, biliverdin, bilirubin and hemoglobin rich structures. Examples of targets for the treatment of non-pigmented structures, tissues and conditions can include, but are not limited to, hair follicles, hair shafts, vascular lesions, infectious conditions, sebaceous glands, acne, and the like.

Methods of treating various skin conditions, such as for cosmetic purposes, can be carried out using the systems described herein. It is understood that, although such methods can be conducted by a physician, non-physicians, such as aestheticians and other suitably trained personnel may use the systems described herein to treat various skin conditions with and without the supervision of a physician.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Treatment devices that rely on applications such as fractional resurfacing use a wavelength that is strongly absorbed by water, which requires accurate delivery of laser energy to maintain treatment density as the treatment device is moved across skin. Excessively high energy density can result in tissue damage, such as burning, which is undesirable for effective treatment. To avoid delivering excess energy to target tissue, treatment devices can possess motion-tracking abilities to record the relative motion of the treatment device and to regulate the energy delivered to the target tissue based on that relative motion of the treatment device.

The relative velocity of typical treatment devices with respect to target tissue, such as skin, can be determined using an optical motion tracking technology like the kind used in optical computer mice. Such trackers have internal sources of infrared (IR) light and an array of photodetectors that detect changes in light intensity over time and can extract motion information from these detected changes. This technology can work well for treatment devices that do not rely on direct contact with the target tissue, but certain advantages are lost when relying on such devices. Treatment devices that rely on direct contact with target tissue can simultaneously treat and cool tissue using a chilled optical window, however the direct contact of the target tissue by an optical window generally tends to flatten out the target tissue and eliminate the presence of certaintopographical variations thereof that would be used for the above-described motion tracking technology.

Further, treatment devices that do rely on direct contact with target tissue tend to also rely on coupling media, such as various refractive index-matching fluids and/or gels to minimize optical reflections from the interface between the optical window and the target tissue. This minimization of optical reflections tends to improve treatment and imaging abilities of the treatment device, but it also tends to further reduce the visibility of surface features relied on for motion tracking. In other words, there are advantages and disadvantages associated with such direct-contact systems and their ability to properly track motion.

Rather than relying on typical topographical features of the skin that would come with the drawbacks explained above, motion tracking techniques can take advantage of certain tissue markers disposed within or beneath skin in order to determine motion relative to skin using the tissue markers as a guide. Such tissue markers can include both natural and artificial substances. Natural substances can include melanin concentration and/or distribution, blood or vasculature in the tissue, pores, hair follicles, crease lines, wrinkles, and other areas providing enough optical variation to distinguish motion relative to the tissue. Artificial substances can include inks and/or dyes applied to the tissue surface or within the tissue. Such artificial substances can be applied in a pattern, for example, and they may act as markers under excitation from certain wavelengths of light. In a specific example, with the possible exception of a case of albinism, melanin is present at some level in all skin types. This makes melanin a good choice as a tissue marker with which to track motion of a treatment device. However, other tissue markers described herein may be suitable as well, depending upon the treatment, patient, etc.

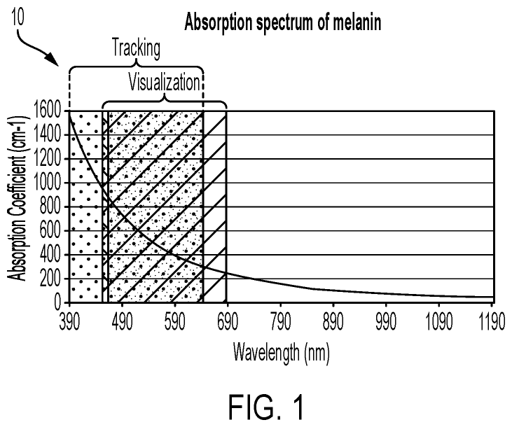

IR light (nominally 850 nm) is poorly absorbed by melanin, as can be seen in the chart 10 of FIG. 1. Instead, IR light tends to diffuse several millimeters into the skin. With no intrinsic chromophore that specifically absorbs IR light, backscattered light is detected only as background light without providing information regarding any features of the skin. As the wavelength of light increases, the absorption coefficient of melanin decreases. Accordingly, certain wavelengths of light are more suitable for motion tracking and visualization in a treatment device. Namely, wavelengths suitable for motion tracking, as shown in the chart 10, are between about 390 nm and about 650 nm. Wavelengths suitable for visualization are between about 450 nm and about 690 nm. UV light from about 315 nm to about 400 nm, as well as light at about 420 nm, are undesirable generally because of their action spectrum of actinic keratosis. Even though small doses at these wavelengths may generally be safe, prolonged exposure can be damaging.

The present disclosure provides systems and devices that address the above-described challenges. In general, high numerical aperture (NA) optical treatment systems are described that can focus electromagnetic radiation (EMR) (e.g., a laser beam) to a treatment region in a tissue. The focused laser beam can deliver optical energy to the treatment region without harming the surrounding tissue. The delivered optical energy can, for example, treat tissue in a treatment region of the dermal layer of the skin, without affecting the surrounding regions (e.g., overlying epidermal layer, other portions of the dermal layer, and the like). In other implementations, the delivered optical energy can cause tattoo removal or alteration, or hemoglobin-related treatment.

Exemplary methods and devices for treating skin conditions with light or optical energy are disclosed in U.S. Patent Publication No. 2021/0138261, entitled “Feedback Detection for a Treatment Device,” U.S. Patent Application Publication No. 2016/0199132, entitled “Method and Apparatus for Treating Dermal Melasma,” and U.S. Provisional Application No. 62/438,818, entitled “Method and Apparatus for Selective Treatment of Dermal Melasma,” each of which is hereby incorporated by reference herein in their entirety.

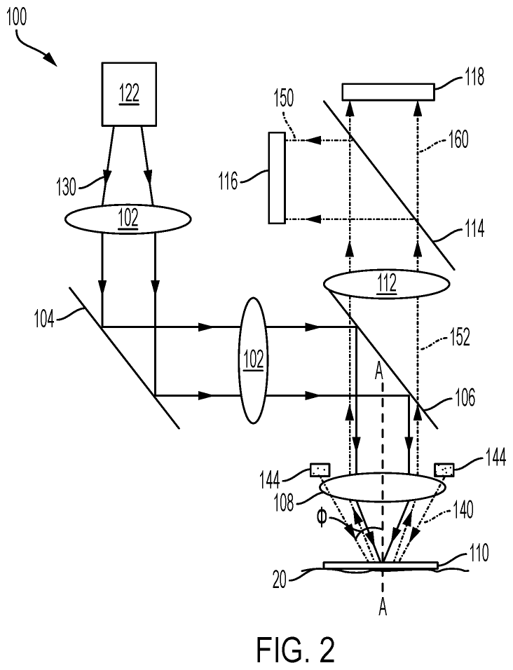

Referring now to FIGS. 2-5, a device for radiative treatment 100 and elements thereof are shown. With these above challenges in mind, the treatment device 100 is designed to include features that either directly address these challenges or that are compatible with certain specialized techniques, such that the techniques can directly address these challenges. For example, the treatment device 100 can generally be an apparatus with compact optical geometry that provides substantially oblique illumination for motion tracking during laser treatment using a high numerical aperture (NA) lens proximate an optical window that is in direct contact with target tissue during at treatment.

The device 100 can generally include optical components that define various optical channels through the treatment device 100 and along which optical energy can travel. In general, the optical components of the treatment device 100 can include one or more of various beam-shaping elements 102 (e.g., axicons, lenses, a combination of reflective and refractive optics, etc.), one or more reflective elements 104, a dichroic beam splitter 106, a high numerical aperture (NA) lens 108, the optical window 110, an imaging lens, a beam splitter 114, an imaging camera 116, and a motion tracker camera 118.

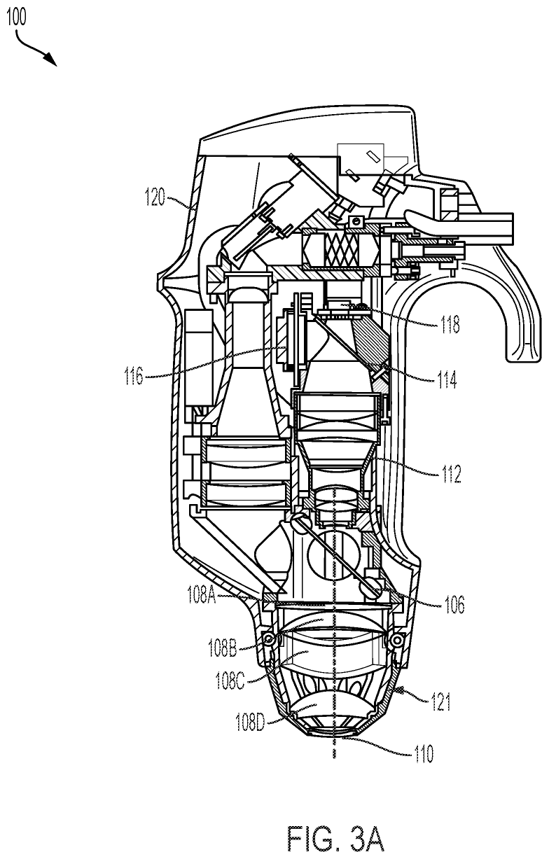

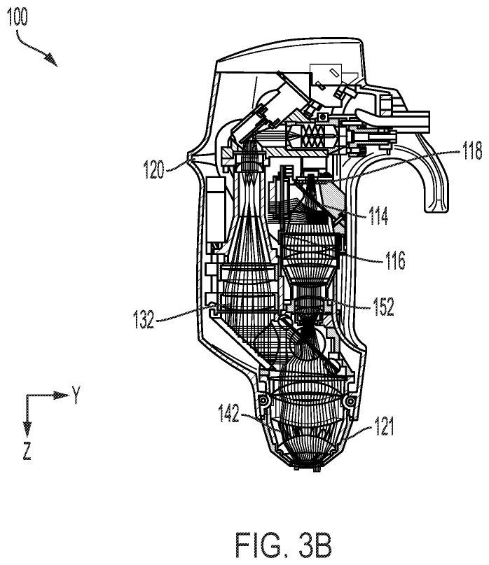

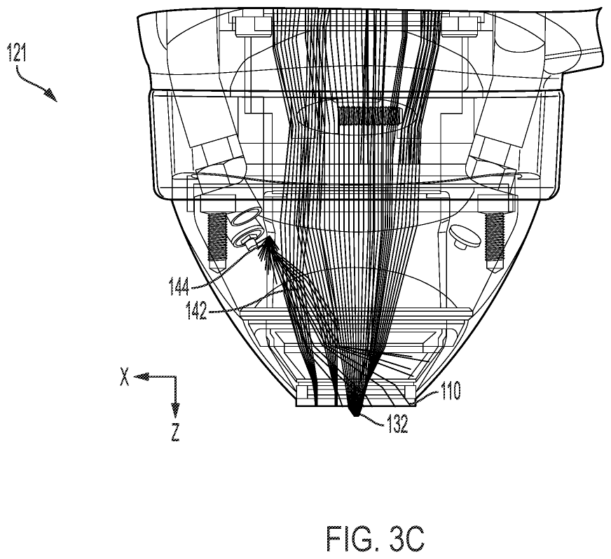

The optical components of the treatment device 100 can be contained in a generally elongate housing 120, which can be seen in FIG. 3A, for example. The housing 120 can be shaped to fit substantially with a clinician's hands such that the treatment device 100 (also generally referred to as a handpiece) can be conveniently maneuvered during a treatment procedure. The optical window 110 can be located at a distal end of the housing 120 in a distal portion of the treatment device 100 referred to as a snout 121. The high NA lens 108 can also be located in the snout 121 of the treatment device, and in some variations, the high NA lens 108 can be more than one lens. For example, as seen in FIG. 3A, the high NA lens 108 featured in FIG. 2 can comprise four (or more or less) lenses 108A-108D located within the snout 121. Together, the high NA lens 108 and the optical window 110 can define an optical axis A-A that generally passes through respective midpoints thereof, and along which at least a portion of the optical channels are defined

The various optical channels can include: a treatment channel 130 along which treatment optical energy 132 can travel; an illumination channel 140 along which illumination optical energy 142 can travel; an imaging channel 150 along which backscattered light 152 can travel to be used to image the target tissue 20; and a tracking channel 160 along which the backscattered light 162 can be used to track motion of the treatment device 100 relative to the target tissue 20. Each of these channels can be seen in the schematic diagram of the device 100 depicted in FIG. 2.

The treatment channel 130 can be defined by the optical components responsible for the transmission and direction of treatment optical energy 132 generated by a treatment light source 122 to a target tissue 20 in order to treat the target tissue 20. Depending upon the demands of a treatment regimen, a wavelength of the treatment optical energy 132 can be between about 1040 nm and about 2020 nm. These optical components can include the one or more of various beam-shaping elements 102 described above, the one or more reflective elements 104, the dichroic beam splitter 106, the high NA lens 108, and the optical window 110.

In operation, treatment optical energy 132 generated by the treatment light source 122 passes through one or more beam-shaping elements 102 where it can be shaped, filtered, collimated, focused, and/or otherwise manipulated as desired, all while being directed by the one or more reflective elements 104 toward the optical window 110 and the target tissue 20, in turn. With respect to the treatment optical energy 132, the dichroic beam splitter 106 can act to reflect the treatment optical energy 132 toward the target tissue 20 without allowing the treatment optical energy 132 to pass through itself. The treatment optical energy 132 can pass through the high NA lens 108, which, in some variations, can have an NA of at least about 0.2, in other variations at least about 0.25, and in further variations at least about 0.3. The treatment optical energy 132 can pass through the optical window 110, which can be a variety of shapes, including being at least partially rounded to various degrees and/or flat. After passing through the optical window 110, the treatment optical energy 132 can interact with the target tissue. The treatment optical energy 132 can be seen in FIG. 3B as it passes through the treatment device 100 along the treatment channel 130. Toward the end of its journey toward the target tissue 20, the treatment optical energy 132 can travel substantially along the optical axis A-A.

The illumination channel 140 can include components responsible for the creation, direction, and transmission of illumination optical energy 142 to illuminate the target tissue 20. These components can include one or more illumination sources 144, the high NA lens 108, and the optical window 110. The one or more illumination sources 144 can be one or more LEDs or other light sources that are able to illuminate the target tissue 20 with illumination optical energy 142 through the high NA lens 108 and the optical window 110. The one or more illumination sources 144 can be disposed within the treatment device 100 near the optical window 110 and offset from the optical axis A-A to avoid interference with the treatment optical energy 132 passing through the treatment channel 130. For example, the one or more illumination sources can emit the illumination optical energy 142 from oblique positions such that an angle φ between the optical axis A-A and the axis of illumination by the one or more illumination sources is at least 33 degrees, as can be seen in FIG. 2. At the angle φ, the illumination at a surface of the target tissue 20 will make an angle in a range of about 0 to 65 degrees. This angle φ can be referred to as a substantially oblique angle, which, in context, means that the illumination optical energy 142 emitted by the one or more illumination light sources 144 can be emitted such that the target tissue 50 is illuminated at a substantially oblique angle. The illumination optical energy 142 can be seen in FIGS. 3B and 3C as it is emitted from the one or more illumination sources 144, passes through the high NA lens 108 and the optical window 110, and reaches the target tissue 20. The one or more illumination sources 144 will be described in greater detail below.

After the illumination optical energy 142 reaches the target tissue 20, at least a portion of it reflects off the target tissue 20 and is scattered into the treatment device 100. The backscattered light 152 passes through the optical window 110 and the high NA lens 108 along the optical axis A-A or substantially parallel thereto, and it then is able to pass through the dichroic beam splitter 106 with minimal interaction therewith. After passing through the dichroic beam splitter 106, the backscattered light 152 can pass through an imaging lens 112 that can be designed to work over broad wavelengths between about 390 nm and about 2020 nm. The imaging lens 112 can have a NA of at least about 0.04, and in some variations, the NA of the imaging lens 112 can be at least about 0.06. As explained above, the thickness of a typical adult epidermis falls in a range of about 50 μm to about 120 μm. The imaging lens 112 can be designed to have a depth of focus range compatible with any epidermis, especially in this typical thickness range. The imaging lens 112 can have an adjustable iris to optimize its F/#in order to improve contrast, as well.

To determine an appropriate f-number for the imaging lens, the following information can be used. For example, at marginal focus and quarter wave of spherical aberration, 4λ(F/#)²≈50 μm to 120 μm, which corresponds to an f-number between about 5.2 and 8.2 at an imaging wavelength of the one or more illumination sources of about 450 nm. As also explained above, and as elaborated on below, the illumination wavelength can be selected depending upon a melanin concentration in the target tissue 20 as well as any other tissue marker.

After passing through the imaging lens 112 (or lenses), the backscattered light 152 can interact with a beam splitter 114, which act to evenly divide the backscattered light between the imaging channel 150 and the tracking channel 160. Prior to this division, the imaging channel 150 and the tracking channel 160 are the exact same.

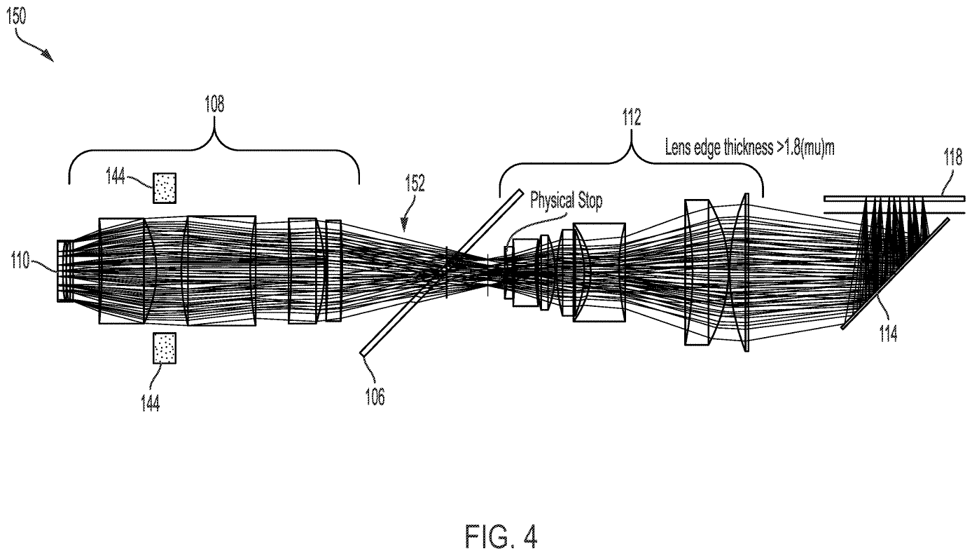

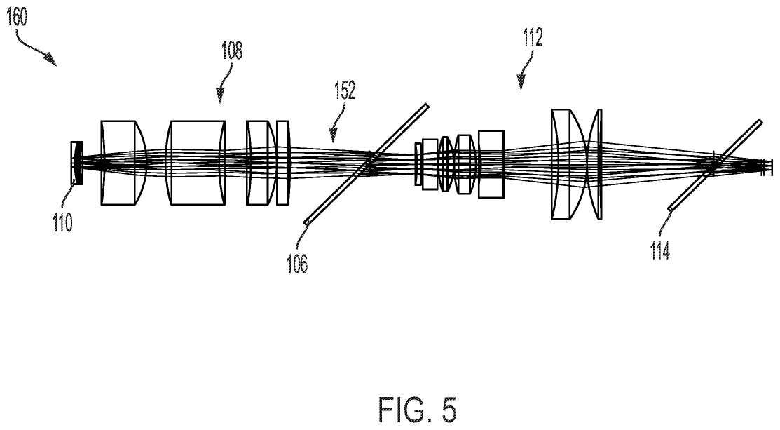

The backscattered light 152 diverted by the beam splitter 114 to the imaging channel 150 can reach an imaging camera 116 located within the treatment device 100, which can be used to image the target tissue 20 during a treatment operation. The imaging channel 150 is depicted FIG. 4. The light diverted by the beam splitter 114 to the tracking channel 160 can reach a motion tracking camera 118, which can be used to track a motion of the treatment device 100 relative to the target tissue 20. The tracking channel 160 is depicted in FIG. 5. Both the imaging camera 116 and the motion tracking camera 118 will be described in greater detail below.

In operation, the treatment device 100 can use the one or more illumination sources 144 operating at different wavelengths of light from about 390 nm to about 650 nm to illuminate the target tissue 20. Backscattered light 152 like the kind described with respect to the imaging channel 150 and motion tracking channel 160 can be used to detect motion of the tracking device 100 relative to the target tissue 20 as the treatment device 100 is moved during a treatment procedure. The efficacy of motion tracking can be improved in a number of ways, including by selecting proper positioning of the one or more illumination sources 144 relative to the high NA lens 108 and optical window 110, and selecting an appropriate wavelength of light emitted by one or more illumination sources 144. Both of these aspects will be taken up in turn, below.

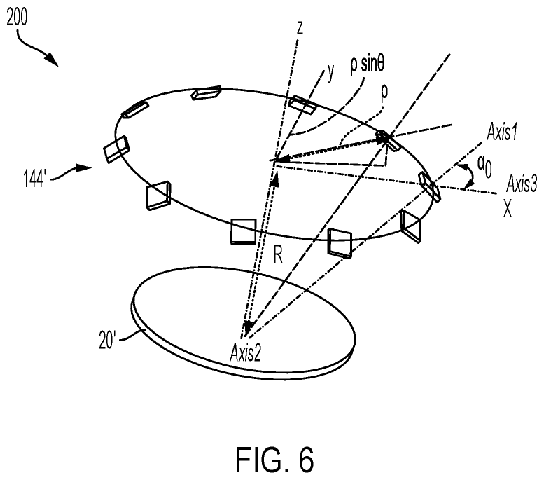

As explained above, the high NA lens 108 can be multiple lenses 108A-108D, as depicted in FIG. 3A. Depending upon the qualities of the high NA lens 108 and/or the multiple lenses 108A-108D, the ideal position of the one or more illumination sources 144 can change. To calculate an initial position and orientation of the one or more illumination sources 144 around a desired lens component (e.g., high NA lens 108, and/or the multiple lenses 108A-108D) the following method can be used in conjunction with the diagram 200 depicted in FIG. 6. FIG. 6 depicts target tissue 20′ and several illumination sources 144′ positioned in a ring-like pattern above the target tissue, which are easily comparable to both target tissue 20′ and the one or more illumination sources 144′.

To begin the method, the desired lens component around which to place the one or more illumination sources 144′ can be determined. By way of example, the lens 108C can be selected as the optical element around which the one or more illumination sources 144′ will be placed. Next, a desired number of illumination sources 144′ can be selected. For tracking, a single illumination source is enough, but for visualization and imaging, more than one illumination source may be helpful to improve uniformity of the illumination at the surface of the target tissue. Then, vary R, p, and ao, where R is the distance from the target tissue 20′, p is the distance to each illumination source 144′ from a center of the lens, and ao is the angle each illumination source normal makes with the target tissue 20′. This is calculated as a tangent to a sphere having a radius R at aperture ρ. θ is defined as the angle between p and the x-axis in the z-plane. Position and rotation angles of any illumination source placed around that sphere can be calculated as:

$$tan {a}_{x} = tan {a}_{0} * cos θ$$

$$tan {a}_{y} = tan {a}_{0} * sin θ$$

$$( x , y ) = ( ρ cos θ , ρ sin θ )$$

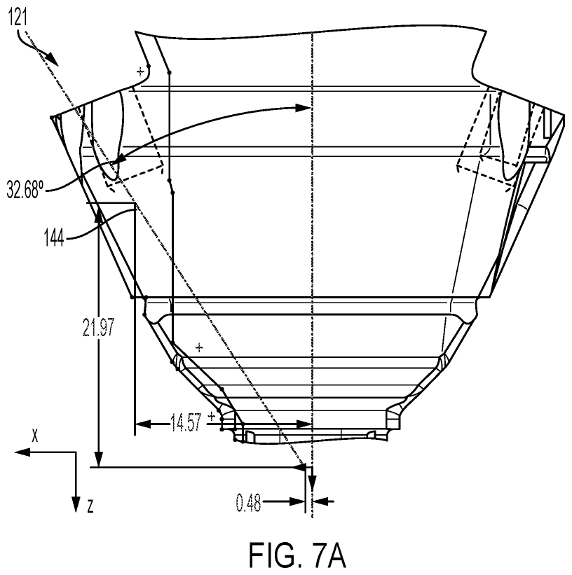

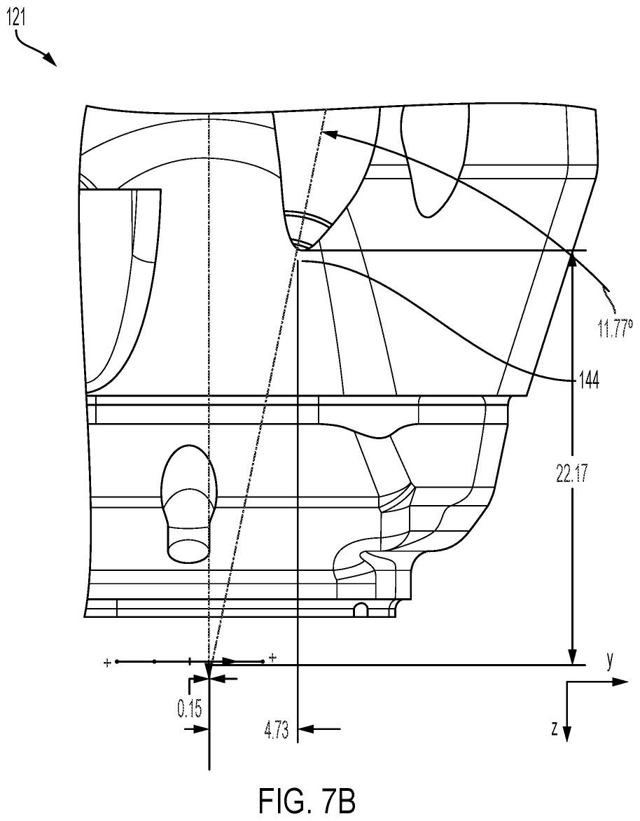

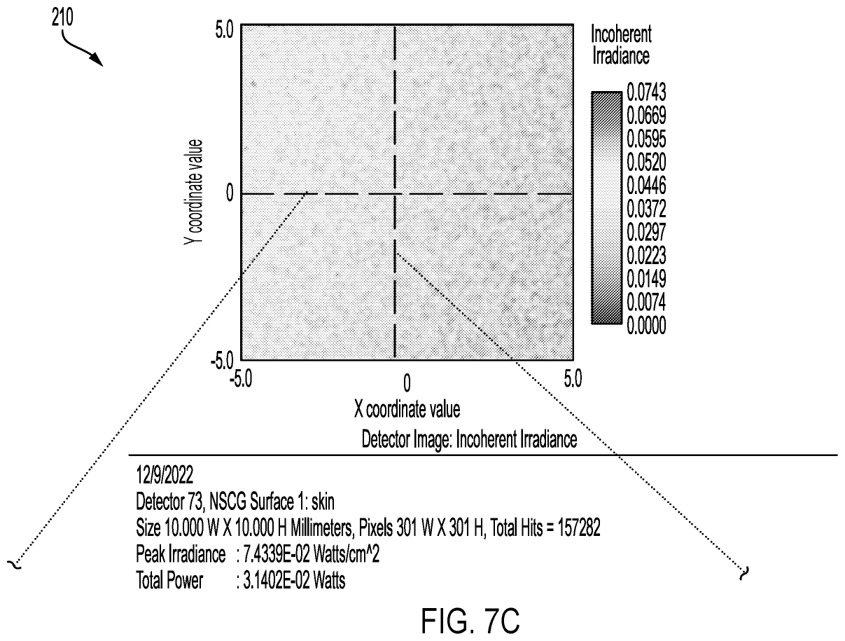

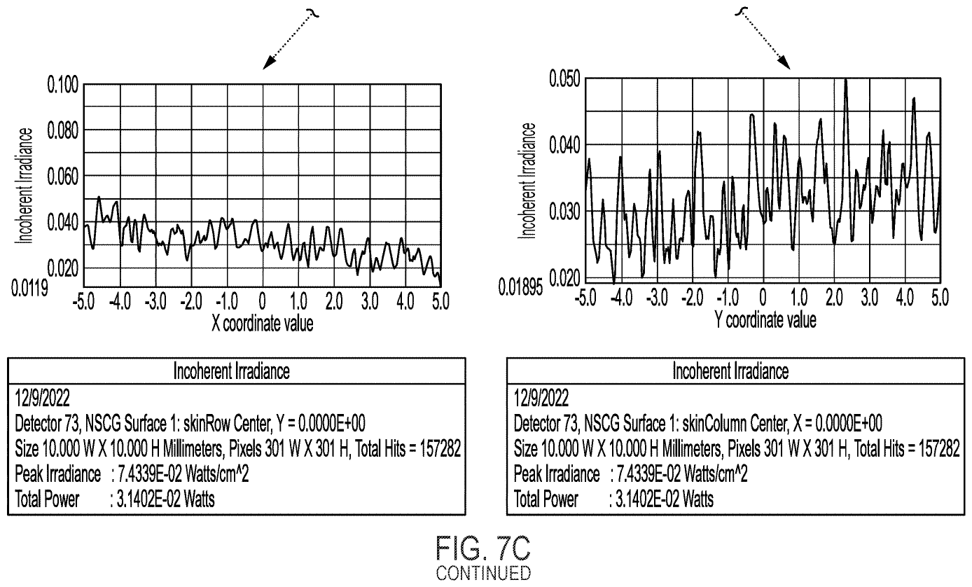

FIGS. 7A and 7B depict a CAD model of the snout 121 showing the optimal parameters of the illumination source 144 used in motion tracking placed around the lens 108C (not shown in the FIG.) in the snout 121 using the above method and calculations. The physical coordinates, of the illumination source 144, with an origin placed at a surface of the target tissue 20′ proximate a center of the optical window 110, are:

$$( x , y , z ) = ( 14.57 mm , 4.73 mm , 22.17 mm )$$

The illumination source 144 can be angled accordingly:

$$( {a}_{x} , {a}_{y} ) = ( 117 ° , 27.72 ° ) .$$

FIG. 7C depicts an optics studio simulation 210 of illuminance at a surface of the target tissue 20 by the illumination source 144 using the parameters described with respect to FIGS. 7A and 7B, and where the illumination source 144 is a blue LED.

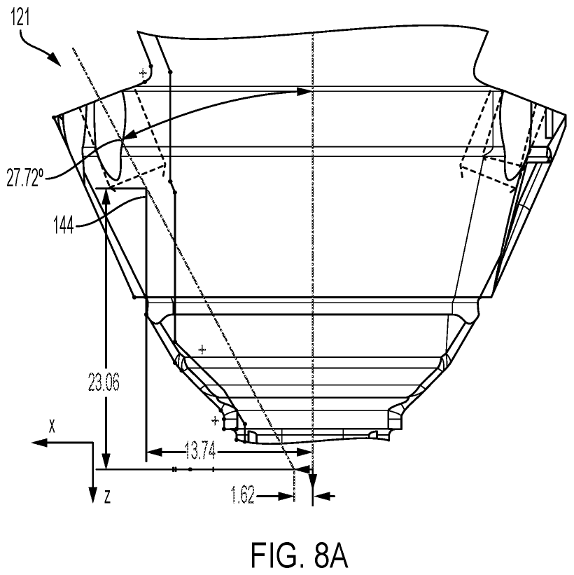

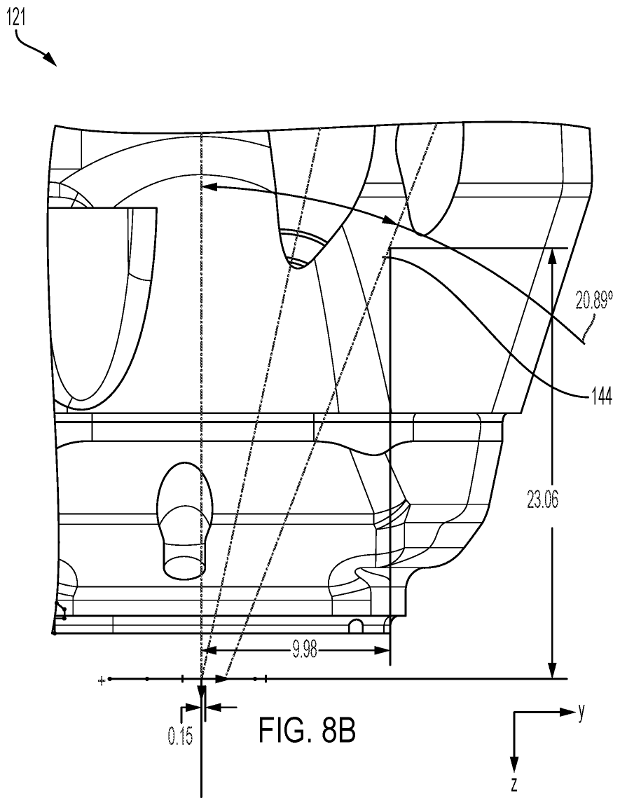

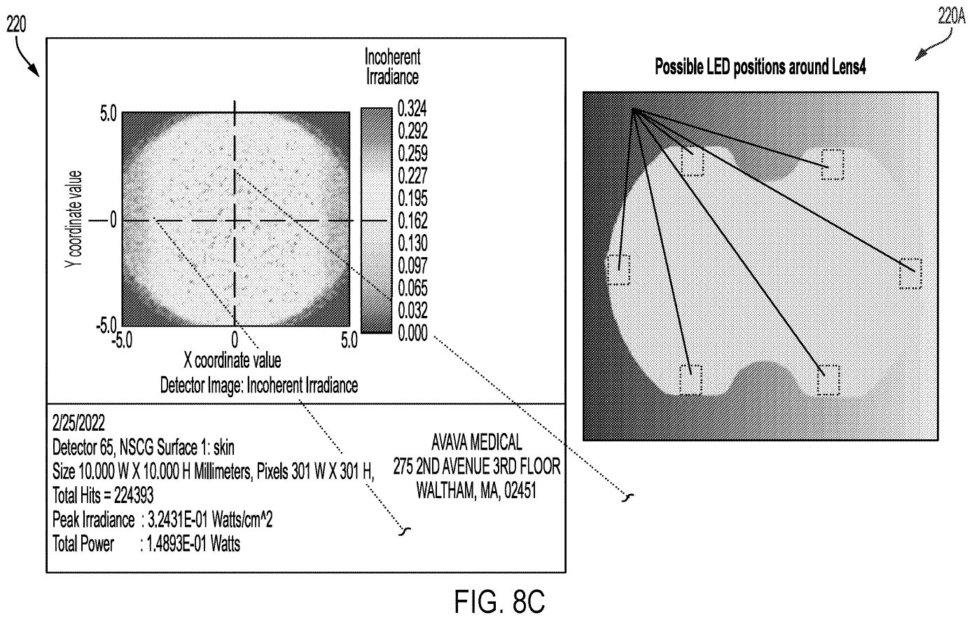

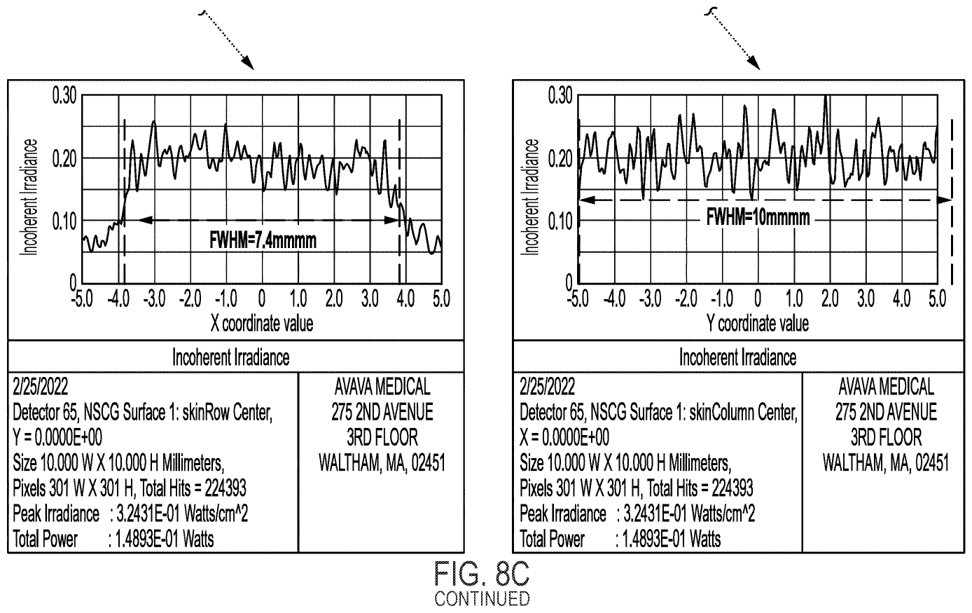

FIGS. 8A and 8B depict a CAD model of the snout 121 showing the optimal parameters of the illumination source 144 used for imaging placed around the lens 108C in the snout 121 using the above method and calculations. The physical coordinates of the illumination source 144 are:

$$( x , y , z ) = ( 13.74 mm , 9.98 mm , 23.06 mm )$$

The illumination source 144 can be angled accordingly:

$$( {a}_{x} , {a}_{y} ) = ( 20.89 ° , 27.72 ° ) .$$

FIG. 8C depicts an optics studio simulation 220 of illuminance at a surface of the target tissue by the illumination source 144 using the parameters described with respect to FIGS. 8A and 8B, and where the illumination source 144 is a white LED. The inset of FIG. 8C depicts alternate positions of the illumination source 144.

Depending upon the kind of lens or lenses used in the treatment device 100, the optimal placement of the one or more illumination sources 144 can change, which can be seen in the inset 220A. The illumination source 144 can be mounted to a metal core printed circuit board (PCB) (not pictured) for proper control of the position and orientation thereof. The metal core can help to conduct heat away from the illumination source 144 and into surrounding metal mounts.

As explained above, direct contact by the optical window 110 can smooth out the target tissue 20 and reduce the presence of physical features that could otherwise be relied for motion tracking. To combat this challenge, melanin can be selected as a tracking marker, for example, because melanin is deposited in the target tissue unevenly, and areas with larger concentration of melanin can standout as trackable features. However, melanin as a trackable feature comes with its own challenges, such as a variation in accuracy of motion tracking techniques as a result of differences in the target tissue 20, most notably seen in skin tissue with varying concentrations of melanin. Using the Fitzpatrick classification system for skin types, skin can be grouped based on an epidermal melanin concentration thereof, resulting in six groupings dubbed Fitzpatrick types 1-VI. Fitzpatrick I-IV skin types have epidermal melanosome concentration by volume in the range of about 1.3-43%, which is usually distributed uniformly in healthy skin. Melanin, as a chromophore, absorbs very well in the blue and in the visible range as shown in FIG. 1. However, at higher concentrations of melanin (Skin types V and VI), blue light is strongly absorbed everywhere, making it difficult to find trackable features. For these skin types, a longer wavelength is more desirable, including using amber or white light.

In practice, the selection of a wavelength for tracking in skin types II-V can be challenging when visual confirmation by a practitioner is the sole determination of that wavelength because the spectral reflectivity and scattering by the target tissue can depend on other factors, including ultraviolet light (UV light) exposure, moisture, etc., which may not be readily discernable by visual confirmation. Accordingly, automation of the selection of this wavelength based on a more quantitative approach, like the kind described herein, and improve accuracy in treatment. While reference is made to melanin as the marker upon which tracking relies, melanin is but one such example. Therefore, the techniques and description provided herein can be generalized and applied to other tissue markers.

An operator of the treatment device 100 can move the treatment device on to-be-treated areas of the target tissue while no treatment optical energy 132 is delivered. While the treatment device 100 is being moved, wavelengths of the one or more illumination sources 144 can be cycled while motion tracking performance is recorded at each iteration of the cycle. This process (referred to as a “pre-scan approach”) can be repeated several times for increased measurement accuracy, and an average performance of the various iterations can be recorded. The wavelength or combinations of wavelengths of the one or more illumination sources that performs the best during the process can be used during an actual treatment procedure.

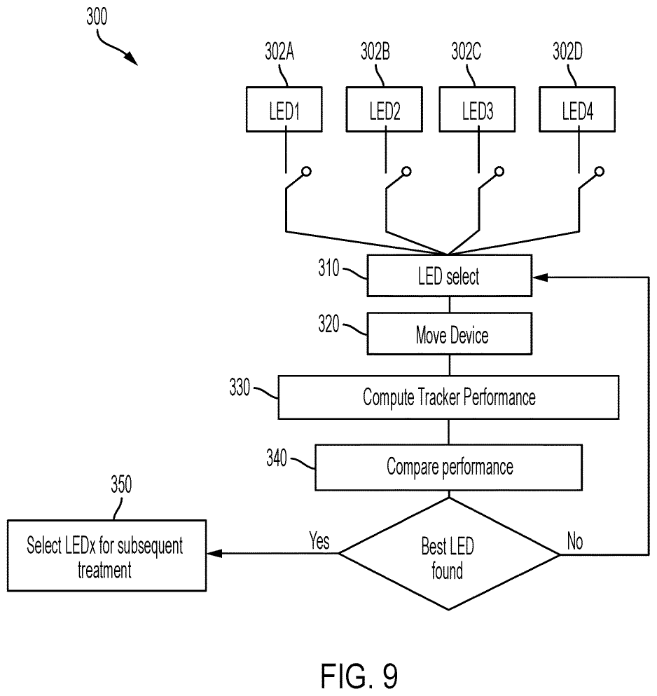

An exemplary method 300 for wavelength selection of the one or more illumination sources can be seen in FIG. 9. In this example, four illumination sources 302A-302D, which can be similar or identical to the one or more illumination sources 144, are being tested. The method 300 can involve several cycles to determine an optimal outcome. In one iteration of the cycle, a certain illumination source (e.g., 302A) is selected at step 310. The treatment device 100 is moved at step 320. Performance is computed at step 330. The computed performance is compared to prior performances at step 340. If the best of the illumination sources 302A-302D is found, that illumination source 302A-302D is used in a treatment at step 350. Otherwise, the process proceeds back to step 310 with a new illumination source 302B-302D.

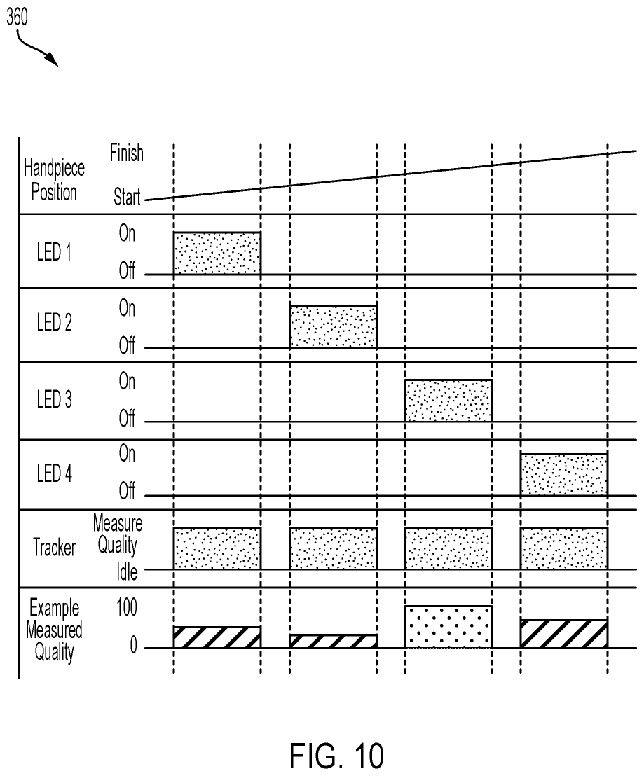

A timing diagram 360 of the method 300 can be seen in FIG. 10, with time proceeding along the x-axis. The treatment device 100 position can be seen in the first row as the method 300 progresses. A power level of a first illumination source 302A can be seen in the second row during the time of the method 300. A power level of the second illumination source 302B can be seen in the third row during the time of the method 300. A power level of the third illumination source 302C can be seen in the fourth row during the time of the method 300. A power level of the fourth illumination source 302D can be seen in the fifth row during the time of the method 300. A measurement state can be seen in the sixth row during the time of the method 300, where measurement coincides with power levels of the illumination sources 302A-302D. An exemplary result of the measured quality of each of the illumination sources 302A-302D can be seen in the seventh row during the time of the method 300. From this result, it can be seen that the third illumination source 302C performed the best and would be selected for use in a treatment procedure.

While the method 300 involves testing a single illumination source at a time, in some variations, combinations of illumination sources could be tested to determined whether multiple illumination sources perform well.

Further variations may rely on a single illumination source with a toggle-able wavelength, i.e., the illumination source can emit a selectable wavelength of light. In such variations, rather than cycling multiple illumination sources, the wavelengths of light that can be emitted by the illumination source can be toggled and measured for quality of performance.

Other methods to determine a wavelength of the one or more illumination sources 144 are contemplated herein. For example, determinations could be made based on a Fitzpatrick skin type using neural networks. Color of the target tissue could be collected in white illumination using a white LED or equivalent illumination source. A CMOS camera built into the treatment device 100 could collect images of the illuminated tissue at various points in time. The collected images could be split into RGB channels and analyzed for spectral signatures of each skin type. This analysis could form the basis for a data set upon which the neural network or machine-learning algorithm could be trained to determine an optimal corresponding wavelength of the one or more illumination sources.

In another example, the one or more illumination sources 144 could briefly emit white light against the target tissue 20 when the treatment device 100 is placed in direct contact with the target tissue 20. The backscattered light 152 could be collected by an onboard camera (e.g., imaging camera 116, motion-tracking camera 118, etc.), and an intensity thresholding algorithm could be used to determine the ideal wavelength for motion tracking, where a lower backscattered intensity corresponds to skin with a higher melanin concentration and will trigger the selection of a longer wavelength.

In another example, the one or more illumination sources 144 could rapidly cycle through available wavelengths of the one or more illumination sources 144. By monitoring an intensity of the backscattered light 152 for each of the wavelengths, a wavelength corresponding to a maximum intensity value could be found quickly.

As explained above, treatment with the treatment device 100 involves depositing treatment optical energy 132 to the target tissue 20 in short bursts, which create microthermal zones (MTZ) of damage in the target tissue 20. A therapeutic dose is on the order of 30-150 mJ. In order to deliver this dose, the treatment optical energy 132 is delivered in pulses lasting for between about 3-10 ms, depending upon the power of the treatment optical energy 132. Normally, motion of the treatment device 100 during a pulse can result in the creation of blurred MTZs, which can result in a reduction of energy density and an increase in size of each MTZ, both of which reduce treatment efficacy.

To address this blurring issue, the treatment device 100 can be equipped with blur compensation hardware 400, which can adjust the delivery of the treatment optical energy 132 in order to minimize or eliminate the negative effects on treatment efficacy associated with blurring.

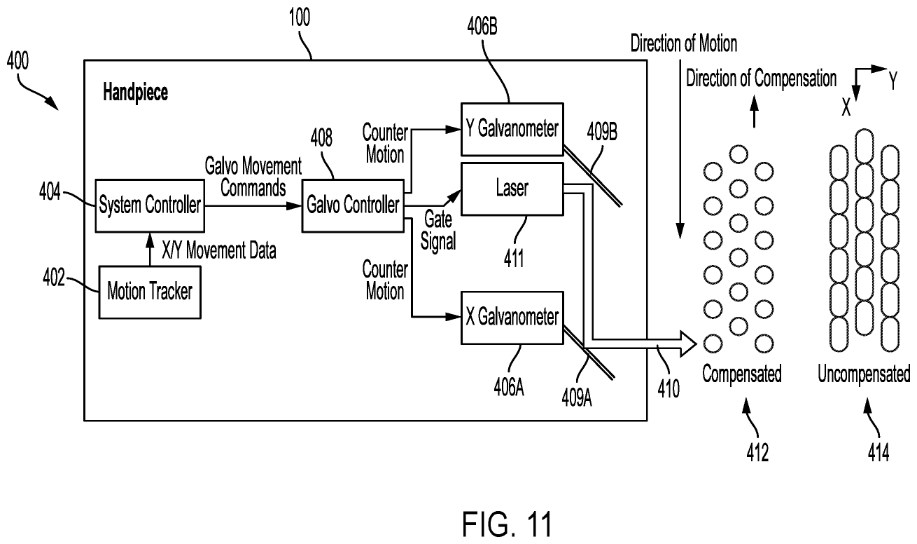

Motion data, like the kind processed as described above for motion tracking, can be fed into a control system within the treatment device 100, which then directs one or more galvanometers in the treatment device 100 to steer the treatment optical energy in a manner that keeps the beam focused one a single spot during the duration of a pulse. A schematic view of the treatment device 100 with the blur compensation hardware 400 can be seen in FIG. 11. The blur compensation hardware 400 includes a motion tracker 402, which feeds X and Y motion data to a system controller 404 for the treatment device 100. The system controller 404 calculates the velocity of the treatment device 100 based on a known conversion factor, determined by the optical elements disposed in the treatment device 100. The calculated velocity can be translated into the necessary commands for movement of one or more galvanometers 406, which can correspond to movement of treatment optical energy (e.g., a laser) about one or more axes of movement. For example, if the treatment device is moved primarily in along an x-axis, the x-galvanometer 406A (i.e., the galvanometer responsible for adjustments relative to the x-axis) can be used to control spacing of the MTZs in the x-axis, as well as to adjust for any motion of the treatment device having a x-component. A y-galvanometer 406B can operate similarly as the x-galvanometer with respect to the y-axis. The resulting compensation can translate into a treatment pattern 412 having discrete, circular MTZs, as opposed to an uncompensated treatment pattern 414 having elongated and blurred MTZs.

The system controller 404 can determine the number of trackable features on the target tissue, and then can select an appropriate wavelength of the one or more illumination sources 144 (particularly one tasked with motion tracking) to maximize tracking quality. The SQUAL value can also be used to determine when the treatment device has been lifted from the surface of the skin.

In operation, the system controller 404 can poll the motion tracker 402 at a rate of about 2000 Hz and reads the number of counts the treatment device 100 has moved since the last poll. The number of counts can be converted to millimeters based on the motion tracker's 402 internal resolution setting and the distance between the one or more illumination sources and the surface of the target tissue 20. With each poll, the system controller 404 accumulates the distance traveled in the x direction in a variable until the treatment device 100 has moved a preset pitch, based on the desired treatment density and a randomized factor. An instantaneous velocity vector and blur compensation vector in the negative-x direction are calculated every 50-100 ms. When the treatment device 100 has moved the pitch, the system controller 404 generates a column of points in the y direction. These points serve as the center point of their respective blur compensation movement. The system controller 404 sends commands related to galvanometer movement and laser pulsing to a galvanometer controller 408 (which can be part of the system controller 404). The galvanometer controller 408 interprets the commands and translates them into analog signals understood by the one or more galvanometers 406 and the source for treatment optical energy 132. The adjustments for compensation can be made based on the received signals. In practice, the adjustments can be made to one or more reflective elements 409, which can be electronically linked to the one or more galvanometers 406. For example, the x-galvanometer 406A can be linked to a first reflective element 409A, which can be adjusted to control an emission angle of a treatment optical energy beam 410 generated by a treatment optical energy source 411 about a first axis—the x-axis. The same can be said for the y-galvanometer 406B linked to a second reflective element 409B, which can be adjusted to control an emission angle of the treatment optical energy beam 410 about a second axis—the y-axis. The emission angle can be measured relative to the optical axis A-A of the treatment device 100.

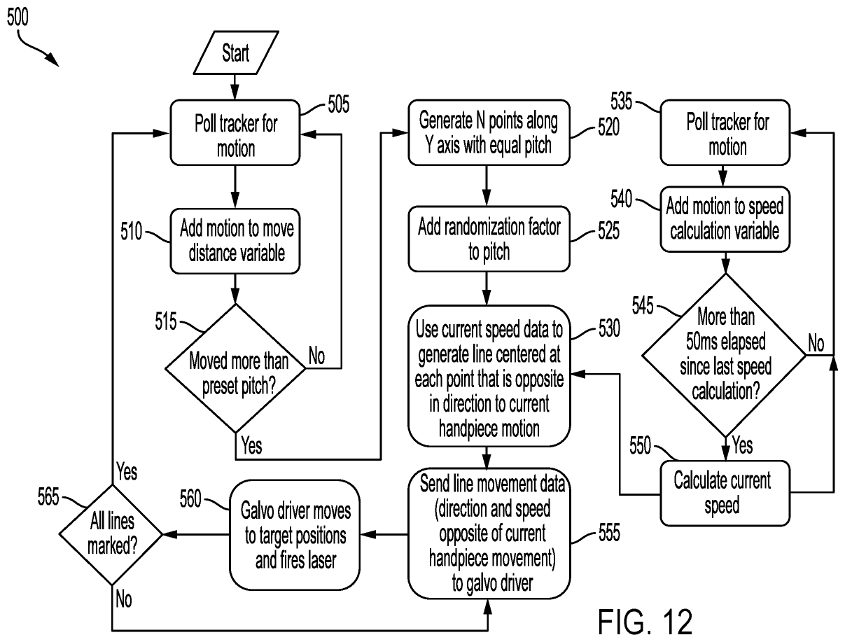

A diagram of the blur compensation process 500 is depicted in FIG. 12. At the beginning of the process 500, the motion tracker 402 can be polled at step 505. At step 510, motion can be added to a move distance variable stored in the treatment device 100. If, at step 515, the treatment device 100 did not move more than the preset pitch, step 505 and step 510 can be repeated. Otherwise, N points can be generated along the y-axis with equal pitch at step 520. Then, a randomization factor 525 can be added to the pitch. Using a current speed of the treatment device 100, a line centered at each point can be generated at step 530, and the lines can be opposite in direction to a current motion of the treatment device 100. The current speed of the treatment device 100 can be calculated by first polling the motion tracker 402 for motion at step 535. Motion can be added to a speed calculation variable at step 540. If less than 50 ms have elapsed since the last speed calculation, step 535 and step 540 can be repeated. Otherwise, the current speed can be calculated at step 550. Once the lines are generated at step 530, the line movement data, consisting of direction and speed opposite the current treatment device 100 movement, can be sent to the galvo controller 408 at step 555. The galvo controller 408 can cause the one or more galvanometers 406 to move to a target position, and the treatment device 100 can dose target tissue with the treatment optical energy at step 560. If all lines are not marked, step 555 and step 560 can be repeated. Otherwise, the process 500 can return to step 505, and a new iteration of the process 500 can begin.

Certain calculations involved in the process 500 are provided as follows. The length of the blur compensation motion is determined by the current speed of the treatment device 100, the energy per pulse, and the power of the treatment optical energy 132. Dwell time, the amount of time the treatment optical energy 132 must be focused at a given spot to deliver the energy per pulse, is given by:

$$Dwell Time ( s ) = \frac{Pulse Energy ( mJ )}{Laser Power ( mW )}$$

The blur compensation movement can be calculated as follows:

$$Blur Compensation {Length}_{X} ( mm ) = Movement {Speed}_{X} ( \frac{mm}{s} ) * Dwell Time ( s )$$

$$Blur Compensation {Length}_{Y} ( mm ) = Movement {Speed}_{Y} ( \frac{mm}{s} ) * Dwell Time ( s )$$

Finally, the velocity vectors of the blur compensation movements are given by:

$$Blur Compensation {Velocity}_{X} ( mm / s ) = - 1 * Movement {Speed}_{X}$$

$$Blur Compensation {Velocity}_{Y} ( mm / s ) = - 1 * Movement {Speed}_{Y}$$

The blur compensation movement and velocity vectors for each of x and y are used to position and move their respective galvanometer. This movement results in the generation of circular MTZs on the target tissue 20 instead of a smeared spot due to the motion of the treatment device 100.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory (“ROM”) or a random access memory (“RAM”) or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input. Further, such devices can include mobile devices, including smartphones, tablets, or other such systems via a website, application, or other program, and user input can be received via a keyboard, touchscreen, stylus, cursor, mouse, etc.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. “Approximately,” “substantially,” or “about” can include numbers that fall within a range of 1%, or in some embodiments within a range of 5% of a number, or in some embodiments within a range of 10% of a number in either direction (greater than or less than the number) unless otherwise stated or otherwise evident from the context (except where such number would impermissibly exceed 100% of a possible value). Accordingly, a value modified by a term or terms, such as “about,” “approximately,” or “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members are present in, employed in, or otherwise relevant to a given product or process. Furthermore, it is to be understood that the disclosed embodiments provide all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the disclosed embodiments where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the disclosed embodiments, or aspects of the disclosed embodiments, is/are referred to as comprising particular elements, features, etc., certain embodiments of the disclosure or aspects of the disclosure consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. For example, any one or more active agents, additives, ingredients, optional agents, types of organism, disorders, subjects, or combinations thereof, can be excluded.

Where ranges are given herein, embodiments of the disclosure include embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. It is also understood that where a series of numerical values is stated herein, the disclosure includes embodiments that relate analogously to any intervening value or range defined by any two values in the series, and that the lowest value may be taken as a minimum and the greatest value may be taken as a maximum. Numerical values, as used herein, include values expressed as percentages.

Although a few variations have been described in detail above, other modifications or additions are possible.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A treatment system, comprising:
a handpiece having a window disposed at an end thereof, the window being configured to directly contact tissue;
a first electromagnetic radiation (EMR) source in communication with the handpiece, the first EMR source being configured to emit a pulsed treatment radiation along a nominal optical axis through the window;
a second EMR source disposed within the handpiece, the second EMR source being configured to illuminate contacted tissue at an illumination angle substantially oblique to the nominal optical axis;
an optical arrangement disposed within the handpiece, the nominal optical axis extending through the optical arrangement;
a first galvanometer in communication with the optical arrangement, the first galvanometer being configured to adjust a configuration of the optical arrangement; and
a controller in communication with the handpiece and the first EMR source, the controller being configured to manipulate the configuration of the optical arrangement via the first galvanometer to alter an emission angle of the pulsed treatment radiation relative to the nominal optical axis and during a treatment pulse duration of the pulsed treatment radiation, the controller being configured to alter a wavelength of the second EMR source based on a detected tissue marker of contacted tissue.

2. The treatment system of claim 1, wherein the second EMR source comprises a plurality of second EMR sources each at a substantially oblique angle relative to the nominal optical axis.

3. The treatment system of claim 2, wherein the plurality of second EMR sources are disposed in a substantially ring-shaped geometry surrounding the nominal optical axis.

4. The treatment system of claim 2, wherein each of the plurality of second EMR sources is configured to emit light at a discrete wavelength, and wherein at least two sources in the plurality of second EMR sources are configured to emit light at different wavelengths.

5. The treatment system of claim 1, wherein the optical arrangement includes an imaging lens configured to receive backscattered light emitted by the second EMR source reflected off the contacted tissue.

6. The treatment system of claim 5, wherein the controller is configured to identify the tissue marker based on the received backscattered light.

7. The treatment system of claim 5, wherein the imaging lens includes an adjustable iris configured to optimize an f-number of the imaging lens.

8. The treatment system of claim 5, wherein the f-number of the imaging lens ranges between about 5.2 and 8.2.

9. The treatment system of claim 5, wherein, at marginal focus and quarter wave of spherical aberration, 4λ(F/#)² is approximately equal to between 50 μm-120 μm,
wherein:
λ is the one or more discrete wavelengths in nm, and
(F/#) is the f-number of the imaging lens.

10. The treatment system of claim 1, wherein the optical arrangement includes a high NA lens having an NA of 0.3 or greater.

11. The treatment system of claim 1, wherein the first galvanometer is configured to manipulate the optical arrangement to adjust the emission angle based on a velocity of the window relative to the contacted tissue.

12. The treatment system of claim 11, wherein the first galvanometer is configured to manipulate the optical arrangement to adjust the emission angle in at least two degrees of freedom.

13. The treatment system of claim 1, further comprising:
a second galvanometer in communication with the optical arrangement,
wherein the first galvanometer is configured to adjust the configuration of the optical arrangement to alter the emission angle about a first axis and the second galvanometer is configured to adjust the configuration of the optical arrangement to alter the emission angle about a second axis, the first axis being substantially perpendicular to the second axis.

14. A treatment system, comprising:
an elongate housing defining a central longitudinal axis, the elongate housing including a window disposed on the housing and intersecting the central longitudinal axis, the window being configured to contact tissue during a treatment process;
an EMR source operably coupled to in the elongate housing and configured to emit a treatment beam toward the window, the treatment beam being emitted along a treatment path that is substantially parallel to the central longitudinal axis;
at least one light source disposed in the elongate housing and radially offset from the central longitudinal axis, the at least one light source being configured to emit a detection beam toward the window;
an optical arrangement disposed within the elongate housing, the optical arrangement being configured to direct and shape the emitted treatment beam;
a first galvanometer in communication with the optical arrangement, the first galvanometer being configured to adjust a configuration of the optical arrangement; and
a controller in communication with EMR source and the first galvanometer, the controller being configured to manipulate the configuration of the optical arrangement via the first galvanometer to alter an emission angle of the treatment beam relative to the longitudinal axis, the controller being configured to alter a wavelength of the detection beam based on a detected tissue marker of contacted tissue.

15. The treatment system of claim 14, wherein the wavelength is adjustable between about 390 nm to 650 nm.

16. The treatment system 14, wherein the wavelength is proportional to the detected tissue marker.

17. The treatment system of claim 14, wherein at least one light source comprises a plurality of light sources each disposed at a substantially oblique angle relative to the longitudinal axis.

18. The treatment system of claim 17, wherein the plurality of light sources are disposed in a substantially ring-shaped geometry.

19. The treatment system of claim 17, wherein each of the plurality of light sources is configured to emit light at a discrete wavelength, and wherein at least two light sources in the plurality of light sources are configured to emit light at different discrete wavelengths.

20. The treatment system of claim 14, wherein the first galvanometer is configured to manipulate the optical arrangement to adjust the emission angle based on a velocity of the window relative to the contacted tissue.

21. The treatment system of claim 20, wherein the first galvanometer is configured to manipulate the optical arrangement to adjust the emission angle in at least two degrees of motion.

22. The treatment system of claim 14, wherein the EMR source is configured to deliver the treatment radiation in a pulse duration, wherein the pulse duration ranges between about 3 and 10 ms.

23. The treatment system of claim 14, further comprising:
a second galvanometer operatively coupled to the optical arrangement,
wherein the first galvanometer is configured to manipulate the optical arrangement to alter the emission angle about a first axis and the second galvanometer is configured to manipulate the optical arrangement to alter the emission angle about a second axis, the first axis being substantially perpendicular to the second axis.

24. A method, comprising:
detecting first backscattered light reflected off a first portion of tissue in contact with a laser treatment device, the backscattered light characterizing respective initial positions of one or more points of interest in the first portion relative to the laser treatment device;
moving the laser treatment device from the first portion of the tissue to a second portion of the tissue;
detecting second backscattered light reflected off a second portion of the tissue as the laser treatment device moves from the first portion of the tissue to the second portion of the tissue, the second backscattered light characterizing respective secondary positions of the one or more points of interest in the second portion relative to the laser treatment device;
determining a velocity of the laser treatment device relative to the tissue based upon the respective initial positions and the respective secondary positions of the one or more points of interest;
delivering at least one electromagnetic radiation (EMR) pulse to the tissue with the laser treatment device for a pulse duration while the laser treatment device is moving along the tissue; and
adjusting, based on the determined velocity, an emission angle of the at least one EMR pulse during the pulse duration, the emission angle being defined relative to a nominal optical axis of the laser treatment device.

25. The method of claim 24, wherein the at least one laser pulse delivers between about 30 and 150 mJ to the tissue during the pulse duration.

26. The method of claim 24, wherein the adjusting comprises changing an orientation of an optical arrangement disposed in the laser treatment device using a first galvanometer.

27. The method of claim 24, further comprising:
determining a dwell time based on an amount of energy delivered to the tissue during the pulse duration and a power of the laser treatment device.

28. The method of claim 27, further comprising:
determining a velocity compensation vector based on the velocity of the laser treatment device and the determined dwell time; and
adjusting the emission angle based on the determined velocity compensation vector.

29. The method of claim 27, wherein the velocity compensation vector is determined between about every 50 to 100 ms.
